(19) United States
(12) Patent Application Publication
(10) Pub. No.: US 2013/0230880 A1
(43) Pub. Date: Sep. 5, 2013

(54) DEVICE AND METHOD FOR ANALYSIS OF BIOLOGICAL SPECIMENS

(71) Applicants: Ansgar J. Pommer, Magdeburg (DE); Michael Ganz, Magdeburg (DE); Eike Barschel, Magdeburg (DE); Lars Philipsen, Magdeburg (DE)

(72) Inventors: Ansgar J. Pommer, Magdeburg (DE); Michael Ganz, Magdeburg (DE); Eike Barschel, Magdeburg (DE); Lars Philipsen, Magdeburg (DE)

(73) Assignee: M04 Tech UG (Haftungsbeschraenkt), Magdeburg (DE)

(21) Appl. No.: 13/820,677

(22) PCT Filed: Sep. 1, 2011
(86) PCT No.: PCT/EP2011/065156

§ 371 Date: May 20, 2013

(30) Foreign Application Priority Data
Sep. 3, 2010 (DE) 10 2010 044 283.6

(51) Int. Cl. C12Q 1/02

(52) U.S. Cl. CPC C12Q 1/02
USPC 435/29; 435/288.7

(57) ABSTRACT

The invention relates to a device and a method for analysis of biological specimens. The device has a control unit into which a kit can be inserted that contains markers required for the analysis of a biological specimen and test records needed therefore. The markers and instructions from the test records are transferred from the kit to the control unit and from thence fed to the individual units of the device.

The invention relates to a device for analysis of biological specimens, a kit comprising the markers required for said analysis and a method for analysis of the biological specimens by means of the device and the kit.

Devices and the associated methods for the detection of proteins in cell specimens are known from the state of the art. For that, the specimens are laced with markers having a binding partner with a high affinity to one of the proteins to be detected and a chromophore, for example a fluorophore, the emitted light of which can be detected by means of microscope, for example a wide-field fluorescence microscope. In this way, fluorescence images of the specimen can be obtained the colorization of which is characteristic for the proteins to be detected there. The image data are stored in a database. Subsequently, the image data read from the database can be processed and visualized with an analysis software. In this way, for example the size of the nucleus of a cell can be measured.

If it is now the problem to determine the size of the nuclei of cells of a biological specimen in practice at first the user has to select the markers required to solve this problem. Then, test records have to be determined that are required to solve the given problem using the selected markers. On the one hand one has to set these test records at the corresponding instruments, for example the fluorescence microscope, on the other hand care must be taken with regard to the interlock of the individual elements of a test record that relate to actions which must be made on different instruments. If then by the fluorescence microscope the user gets the desired image data so he has to configure the analysis software such that he can actually take the desired information, i.e. information regarding the size of the nuclei, from the image data. For that, he has to select suitable software routines and check their suitability by means of the test records and the visualized results of this procedure.

Each of the measures that have to be taken by the user to arrive from his analysis problem at the analytical result is time-consuming and error-prone. Here, individual errors can potentiate which can result in wrong analytical results or a failure of the entire analytical method.

Object of the invention is to eliminate the drawbacks according to the state of the art. There is in particular provided a device for the analysis of biological specimens that allows an automation of the analysis of biological specimens. Furthermore, a kit is provided which comprises the markers required for the analysis, and a method for analyzing the biological specimens by means of the device and the kit.

This object is solved by the features of claims 1 and 9. Practical embodiments of the inventions result from the features of the dependent claims.

In accordance with the invention there is provided a kit for use in the analysis of a biological specimen, which at least comprises
(a) one or more marker(s) required for the analysis of the biological specimen as well as, if required, the process aids needed; and
(b) one or more electronically stored test record(s) comprising instructions for performing the analysis of the biological specimen by means of the markers.

Preferably, the kit comprises all the markers required for the analysis of the biological specimen. The kit should comprise all test records required to perform the analysis of the biological specimen.

Further, the kit can comprise
(c) electronically stored software for the evaluation of measuring results obtained from the biological specimen by means of the markers and using the test records.

Moreover, the kit can comprise
(d) one or more electronically stored test record(s) comprising instructions for the preparation of the biological specimen. Also, the process aids required for the preparation are included in the kit.

Preferably, the kit comprises a unit with a number of reservoirs for receiving the markers and process aids (in the following referred to as “receiving unit”) as well as a unit for the electronic storage of the test records and optionally the software (in the following referred to as “memory unit”). Preferably, the memory unit comprises an interface allowing the communication of the memory unit with another electronic unit. Preferably, the receiving unit is a microwell plate. Preferably, the memory unit is a storage medium with a hardware interface. For example, the memory unit can be a data store with USB interface.

In one embodiment of the invention the memory unit is integrated within the receiving unit. For example, the data store with USB interface can be integrated within the microwell plate. In this way, a receiving unit with an integrated memory unit having an interface is obtained.

The receiving unit of the kit contains the markers that are required for the analysis of the biological specimen according to the test records that are electronically stored in the memory unit. Further, the receiving unit can contain the process aids that are required to effect chemical and/or physical binding of the markers to the constituents of the biological specimen to be detected. The individual markers and, if present, the individual process aids are placed in separate reservoirs of the receiving unit. In order to increase the lifetime of a kit the markers and, if present, the process aids are included in the lyophilized form in the reservoirs, for example. Preferably, the reservoirs are closed, for example with a foil that is removed immediately before the kit is used.

Preferably, the receiving unit of the kit is realized as a microfluidics system.

Thus, the kit preferably consists of a mechanical part, the receiving unit, wherein markers and process aids are stored, and an electronic storage medium, the memory unit, wherein all the information required to perform the analysis are provided. In the present invention, said information are referred to as test records.

In the present invention, the term “marker” describes a chemical compound that can react with a chemical or biological unit, for example a protein contained in the biological specimen. Binding of the marker to the chemical or biological unit can be detected by means of analytical methods. For example, a marker can have a binding partner with high affinity to one of the proteins to be detected and on the other hand a chromophore, for example a fluorophore. The binding partner and the chromophore may be linked to each other via a linker. For example, the binding partner may be a chelating agent, in particular if ions have to be detected in the biological specimen, or an antibody if antigens have to be detected in the biological specimen. When the biological specimen is laced with the marker so the binding of the marker to the chemical and biological unit can be qualitatively or quantitatively detected by exciting the chromophore to emit light. Then, the emitted light can be detected, for example by means of a microscope such as a wide-field fluorescence microscope.

In the present invention, the term “process aid” describes a chemical substance that is required to bring the markers contained in the kit into contact and/or reaction with the biological specimen in the manner given in the test records. Examples of process aids are solvents such as water or isopropanol. When the kit also contains test records with instructions for the preparation of the biological specimen so the term “process aids” also comprises the chemical substances necessary for that.

In the present invention, the term “test record” describes the sum of information and instructions required to be able to analyze the biological specimen by means of the markers using an analyzer. A test record for example comprises
information on the type and amount of the markers contained in the kit;
information on the type and amount of the process aids contained in the kit;
information on the reservoir in which the respective marker or process aid from the receiving unit is located;
instructions to the relative time points for taking each marker or process aid from the respective reservoir of the receiving unit with respect to the beginning of the analytical method;
instructions to the place to which the markers or process aids taken from the receiving unit are to be transferred;
instructions to the residence time of the markers or process aids at this place;
instructions to the basic settings of the individual components of the analyzer at the beginning of the analytical method;
instructions to the control of the individual components of the analyzer after the beginning of the analytical method, for example after receipt of certain markers, process aids, or data;
instructions regarding the type of the image data to be detected;
instructions regarding the type of the analysis to be performed;
instructions regarding the chronological course of the individual steps of the analytical method.

The place may be for example the specimen uptake of the detection unit.

When the kit also comprises test records with instructions for the preparation of the biological specimen so the term “test record” in the present invention additionally comprises the sum of information and instructions required to perform the preparation of the biological specimen by means of the process aids contained in the kit using the analyzer.

In a preferred embodiment, one or more of the following information are stored in the memory unit of the kit:
rules for the application of the markers and process aids to the specimen (information on the order, concentration, exposure time, pre and post-treatment, calibration, etc.),
rules on the control of the detection unit (microscope or other),
rules on the performance of the actual measurement (picture taking with parameters such as exposure time, required wave lengths, filter, picture taking positions, etc.),
rules on the adaptive affection of the above-mentioned parameters for the purposes of optimization and quality assurance during the measuring process,
operators for the execution of the above-mentioned rules,
operators for the control of peripherals,
numerical operators for the evaluation, analysis, and presentation of the measured data and analytical results collected therefrom,
software and software updates for the control system.

In accordance to the invention there is further provided a control system for controlling the analysis of a biological specimen that comprises a data processing unit and a unit for receiving a kit. The control system further comprises
an interface allowing the communication between the data processing unit and the kit;
an interface allowing the communication between the data processing unit and a transfer unit for the transfer of markers and, if present, process aids from the kit to a detection unit; and
an interface allowing the communication between the data processing unit and the detection unit to generate image data of the biological specimen.

Preferably, the control system further comprises one or more of the following interfaces:
an interface allowing the communication between the data processing unit and an analysis unit for the analysis of the image data obtained by means of the detection unit to generate analytical data;
an interface allowing the communication between the data processing unit and a data base for the storage of image data and/or analytical data; and
an interface allowing the communication between the data processing unit and a visualization unit for the visualization of image data and/or analytical data.

Preferably, the data processing unit comprises at least one microprocessor, data stores, and input and output units. Preferably, the data processing unit is provided with an operating system that loads and executes the test records provided by the kit such as for example operators and rules. The operating system manages the available hardware of the analyzer and on this hardware starts the processes required for the analytical method. It can be compared to the operating system of a computer.

The analysis unit can be realized by means of the data processing unit of the control system or a separate data processing device.

The unit for receiving a kit enables the user to insert a kit selected by him/her that is suitable for the analysis of a biological specimen into the control system. Here, via the interface of the kit on the one hand and the interface of the control system that allows the communication between the data processing unit and the kit a connection is established by which the test records can be transferred from the memory unit of the kit to the data processing unit of the control system. This ensures that the markers and, if present, the process aids of the kit, are employed as directed which prevents analytical errors, allows an automation of the analysis of biological specimens, and means a chronological and spatial reduction of the analytical effort.

The interface of the memory unit of the kit and the interface of the control system allowing the communication between the data processing unit and the kit are synchronized. If the interface of the memory unit of the kit is a USB interface so the related interface of the control system, i.e. the interface of the control system allowing the communication between the data processing unit and the kit, is also a USB interface. For example, the memory unit of the kit can have a USB plug, the control system a USB socket. Upon insertion of the kit into the facility of the control system for receiving the kit there is at the same time introduced the USB plug into the USB socket. With that the data link between the kit and the control system is made. Moreover, the reservoirs of the kit are in a predetermined position in the control system.

The remaining interfaces of the control system serve for the communication with facilities required for the analysis of the biological specimen. These facilities comprise one or more of the following facilities:
a transfer unit to transfer markers and, if present, process aids from the kit to a detection unit;
a detection unit to generate image data of the biological specimen;
an analysis unit to analyze the image data obtained with the detection unit to generate analytical data;
a data base to store image data and/or analytical data; and
a visualization unit to visualize image data and/or analytical data.

In the following, the interface of the control system that allows the communication with one of these components is referred to as “related interface”. For example, the interface allowing the communication between the data processing unit and a transfer unit is the related interface for communication between the data processing unit of the control system and the transfer unit.

The control system can be formed as a separate instrument and thus, represent an independent device. When the control system is a separate instrument, so in the following it is referred to as controller.

For taking and transferring the markers or process aids the transfer unit can have one or more automatic pipettes. For example, the transfer unit can be a pipetting robot. Preferably, the transfer of the markers and/or process aids is carried out with a microfluidics system.

In accordance with the invention there is further provided an arrangement for the analysis of biological specimens that comprises the controller, a transfer unit, and a detection unit. A kit can be inserted into the controller. Further, the arrangement can comprise at least one of the following facilities: analysis unit, visualization unit, data base. The units are each external devices. In each case, the controller and the individual units of the arrangement can be connected by means of cables.

In accordance with the invention there is further provided an analyzer comprising:
a transfer unit to transfer the markers from the kit to a detection unit, wherein the transfer unit communicates via the related interface of the control system therewith; and
a detection unit to generate image data of the biological specimen, wherein the detection unit communicates via the related interface of the control system therewith.

Furthermore, the analyzer can comprise at least one of the following units:
an analysis unit to analyze the image data obtained by means of the detection unit to generate analytical data, wherein the analysis unit communicates via the related interface of the control system therewith;
a data base to store image data and/or analytical data, wherein the data base communicates via the related interface of the control system therewith; and
a visualization unit to visualize image data and/or analytical data, wherein the visualization unit communicates via the related interface of the control system therewith.

The analyzer according to the invention and the arrangement according to the invention allow the automated, fast, and almost error-free performance of the analysis of biological specimens.

In accordance with the invention there is further provided an analyzer for the automated analysis of a biological specimen that comprises
a control system comprising
   a data processing unit;
      a unit for receiving a kit, wherein the kit comprises a unit (receiving unit) with a number of reservoirs for receiving one or more marker(s) required for the analysis of the biological specimen as well as, if needed, the process aids required therefore as well as a unit (memory unit) for the electronic storage of one or more electronically stored test record(s) comprising instructions for the performance of the analysis of the biological specimen by means of the markers, and wherein the memory unit is integrated within the receiving unit;
      an interface allowing the communication between the data processing unit and the kit;
      an interface allowing the communication between the data processing unit and a transfer unit for the transfer of the markers and process aids from the kit to a detection unit;
      an interface allowing the communication between the data processing unit and the detection unit for the generation of image data of the biological specimen; and
      an interface allowing the communication between the data processing unit and an analysis unit to analyze the image data obtained by means of the detection unit to generate analytical data;
a transfer unit for the transfer of the markers from the kit to a detection unit, wherein the transfer unit communicates via the related interface of the control system therewith;
a detection unit to generate image data of the biological specimen, wherein the detection unit communicates via the related interface of the control system therewith; and
an analysis unit to analyze the image data obtained by means of the detection unit to generate analytical data, wherein the analysis unit communicates via the related interface of the control system therewith.

In accordance with the invention there is further provided a method for analysis of a biological specimen by means of an analyzer or arrangement according to the invention which comprises the following steps:
(a) providing a kit comprising the markers, process aids, test records, and optionally software that are required for the analysis of a given biological specimen;
(b) introducing the kit into the control system and transferring test records stored there into the data processing unit of the control system;
(c) determining the instructions from the test records required for controlling the transfer unit by means of the data processing unit and transferring said instructions via the related interface of the control system to the transfer unit;
(d) determining the instructions from the test records required for controlling the detection unit by means of the data processing unit and transferring said instructions via the related interface of the control system to the detection unit; and
(e) transferring markers and process aids from the kit to the detection unit by means of the transfer unit on the basis of the instructions transferred in step (c); and
(f) generating image data of the biological specimen by means of the detection unit on the basis of the instructions transferred in step (d).

If the analyzer or the arrangement comprise an analysis unit so the method may further comprise the following steps:
(g) determining the instructions from the test records required for controlling the analysis unit by means of the data processing unit and transferring said instructions via the related interface of the control system to the analysis unit;
(h) transferring the image data obtained in step (f) to the analysis unit and analyzing the image data by means of the analysis unit using the instructions transferred in step (e) to obtain analytical data.

It may be provided that in step (g) there is further transferred software that has been transferred from the kit into the data processing unit from the data processing unit to the analysis unit and that in step (h) in addition to the instructions the software for the analysis of the image data is used. The software can comprise certain algorithms that are executed in the analysis unit to obtain information on the biological specimen from the image data.

If the analyzer or the arrangement comprises a database, so the image data and/or the analytical data can be transferred to the data processing unit and there stored in a database.

If the analyzer or the arrangement comprises a visualization unit, so the image data and/or analytical data can be transferred from the data processing unit via its related interface to the visualization unit.

In the following, the invention is explained in more detail by means of examples that should not limit the invention with respect to the drawings. Here

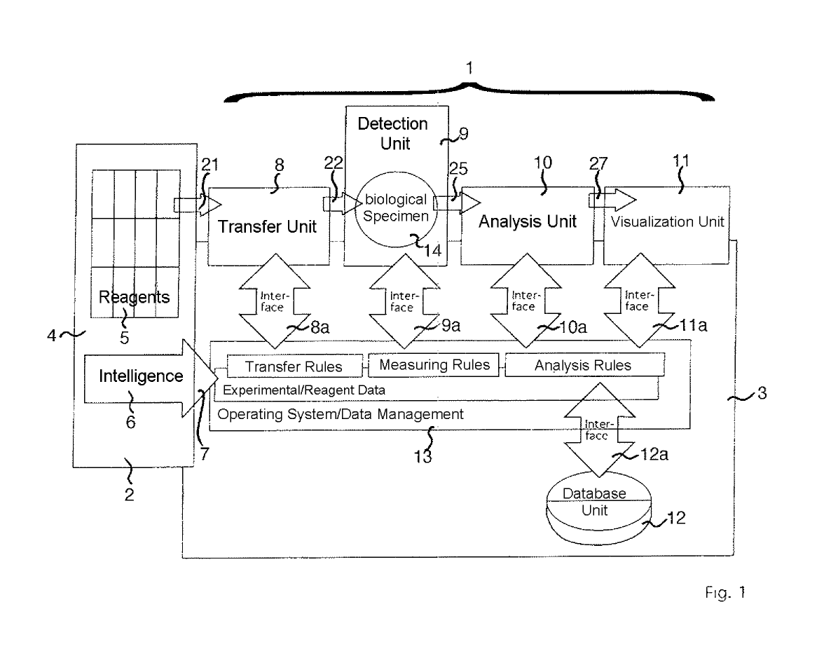

FIG. 1 shows a schematic representation of an embodiment of the analyzer according to the invention with the kit inserted; and

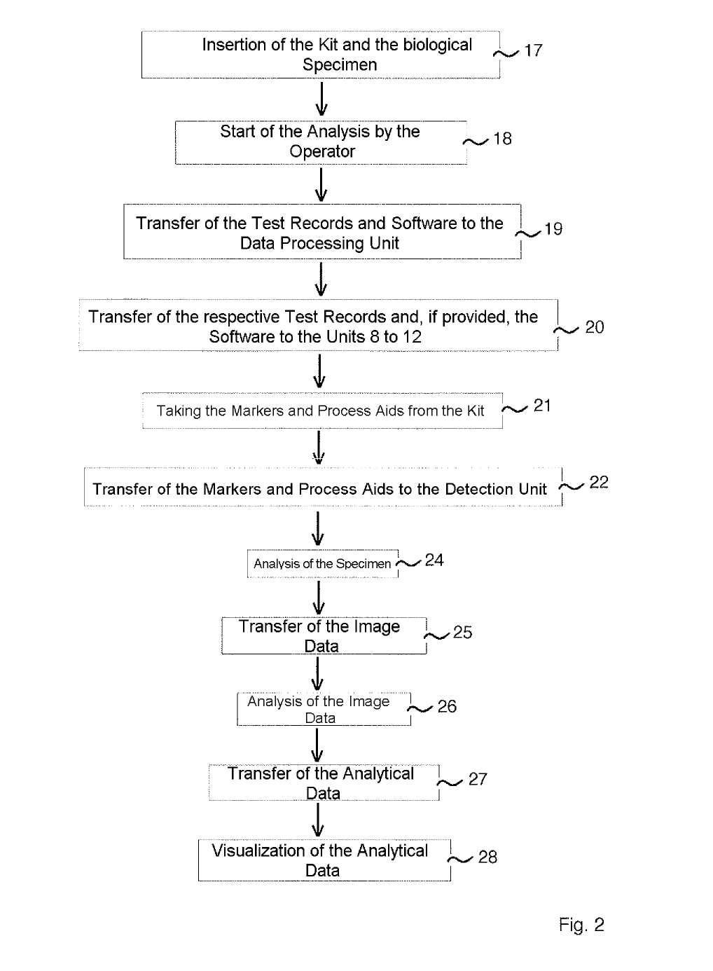

FIG. 2 shows a flowchart representing the course of an embodiment of the method according to the invention by means of the analyzer shown in FIG. 1.

### EXAMPLE 1

### Kit

Hereinafter, a kit is described that can be used to determine the size of the nucleus and the size of the cell of mononuclear cells from the blood and to calculate the size ratio of nucleus to cell. The kit comprises:

(a) Markers and Process aids:
Marker 1 (detection of the nucleus): propidium iodide
   excitation wavelength 536 nm,
   emission wavelength 617 nm,
   concentration: 0.1 μg/ml
   incubation time: 15 min
Marker 2 (detection of the cell membrane): anti CD45 antibody directly linked to Alexa 488 dye
   excitation wavelength 495 nm,
   emission wavelength 519 nm,
   concentration: 1 μg/ml
   incubation time: 15 min
Washing Solution
   PBS (phosphate buffered saline) with 0.5% BSA (bovine serum albumin)
      (b) electronically stored test records:
a rule for the production of the preparation (specimen) for the microscopy
a rule for the application of the markers onto the biological specimen
a rule for the digital picture taking at the wide-field fluorescence microscope inclusive taking calibration images
   (c) electronically stored software for the evaluation of the measuring results
software with algorithms for registration and image normalization and image correction, respectively
software with algorithms for determination of the labeled cell membrane
software with algorithms for determination of the labeled nucleus
software with algorithms for determination of the ratio of size of the nucleus and the whole cell
software with algorithms for statistical evaluation and visualization

### EXAMPLE 2

### Analyzer

Hereinafter, an analyzer is described that can be used to determine the size of the nucleus and the size of the cell of mononuclear cells from the blood and to calculate the size ratio of nucleus to cell. For that, the analyzer 1 includes a control system 3 as well as a transfer unit 8, a detection unit 9, an analysis unit 10, a visualization unit 11 as well as a database 12 for storing image data and analytical data. The control system 3 includes a data processing unit 13 and a unit (not shown) for receiving a kit 2.

The kit 2 comprises a supporting plate 4 on which a number of reservoirs 5 for receiving markers and process aids is provided. On the supporting plate 5 there is further provided a memory unit 6 for storing test records and software. The memory unit 6 has an interface 7 (in FIG. 1 shown in the form of an arrow) which can be brought into contact with the control system 3.

In FIG. 1, the kit 2 is inserted 17 in the receiving unit of the control system 3. With that, the memory unit 6 of the kit 2 is in contact with the data processing unit 13 of the control system 3 via its interface 7. At the same time, the transfer unit 8 of the analyzer 1 has access to the markers and process aids that are in the reservoirs 5 of the kit 2.

The control system 3 has interfaces via which it can communicate with units 8 to 12 as well as with the memory unit 6 of the kit 2. Upon insertion of kit 2 into the receiving unit of the control system 3 and introducing a biological specimen 14 into the detection unit 9 the analytical method can be started by an operator. After start 18 the test records, software and experimental and reagents data stored in the memory unit 6 are fetched 19 by the data processing unit 13 of the control system 3. Subsequently, the test records and software are transferred 20 from the control system 3 to the related units 8 to 11 of the analyzer 1. Here,

(i) the test records (in FIG. 1 referred to as transfer rules) relating to the transfer of the markers and process aids to the specimen and also the residence time of the markers and process aids on the specimen are transferred via interface 8a between the control system 3 and the transfer unit 8 to the transfer unit 8;
(ii) the test records (in FIG. 1 referred to as measuring rules) relating to the performance of the analytical method, i.e. lacing the biological specimen with the markers and the required process aids, and the generation of image data are transferred via interface 9a between the control system 3 and the detection unit 9 to detection unit 9; the test records relating to the performance of the analysis, i.e. the analysis of the cell and the nucleus of the specimen, are transferred via interface 9a between the control system 3 and the detection unit 9 to detection unit 9;
(iii) the test records and the software (in FIG. 1 commonly referred to as analysis rules) relating to the analysis of the image data, e.g. the algorithms for determination of the size of the cell and the nucleus of the specimen, are transferred via interface 10a between the control system 3 and the analysis unit 10 to analysis unit 10; and
(iv) the test records and the software (in FIG. 1 commonly referred to as analysis rules) relating to the visualization of the analytical results and/or the image data are transferred via interface 11a between the control system 3 and the visualization unit 11 to visualization unit 11.

So, units 8 to 11 have all the required data, information, and instructions to automatically perform the analysis of the specimen including the evaluation of the image data and their visualization without further operator intervention. The course of the analysis of the specimen is controlled by the data processing unit 13 of control unit 3. For that, on the basis of a master test record (in FIG. 1 referred to as experimental/reagents data) that is also stored on the memory unit 6 and from thence has been transferred to the data processing facility the data processing unit 13 generates control signals that are transferred via interfaces 8a, 9a, 10a, 11a, and 12a to the units 8, 9, 10, 11, and 12. Also, the units 8 to 12 generate signals confirming the reception of the control signal and the execution of the step designated by the test records. The signals are transferred by the units 8, 9, 10, 11, and 12 also via the interfaces 8a, 9a, 10a, 11a, and 12a. Moreover, the interfaces serve for the transfer of image data and analytical data between the units 8, 9, 10, 11, and 12.

For the generation of the control data as well as the reception and processing of the signals by the units 8, 9, 10, 11, and 12 the data processing unit has a common operating system also allowing data management.

On the basis of the received test records and always after receiving the required control signal the transfer unit takes 21 the given marker or the given process aid from the given reservoir 5 of kit 2 and transfers 22 the taken marker or process aid to the detection unit 9. For taking and transferring the markers or process aids the transfer unit may have one or more automatic pipettes. For example, the transfer unit can be a pipetting robot, preferably, the transfer of the markers and/or process aids is carried out with a microfluidics system.

In the detection unit 9, which for example is a wide-field fluorescence microscope, the biological specimen 14 is analyzed 24 according to the test records transferred there by contacting the specimen 14 with the markers and the required process aids and generating image data. The image data are transferred 25 by the detection unit 9 via interface 9a, data processing unit 13, and interface 10a to the analysis unit 10 and there analyzed 26 according to the test records and software that have been transferred to the analysis unit 10. Finally, the analytical data are transferred 27 by the analysis unit 10 via interface 10a, data processing unit 13, and interface 11a to the visualization unit 11 and can there be observed by the operator 28. Also, the image data can be transferred from the data processing unit 13 to the visualization unit 11. The visualization unit may be a screen or a display.

Furthermore, the image data and analytical data can be transferred from the data processing unit 13 via interface 12a to the database 12. From thence, the image data and analytical data can be again transferred to the visualization unit 11 via interface 12a, data processing unit 13, and interface 11a at any time by request of an operator.

After executing all test records the kit 2 can be removed from the receiving unit of the control system 3 and, if no further analysis of the same specimen is required, the analyzed specimen 14 can be removed from the detection unit 9. With the insertion of a new kit 2′ into the receiving unit of the control system 3 a further analysis of the same or another biological specimen 14 can be started.

### LIST OF REFERENCE NUMBERS

1 analyzer
2 kit
3 control device
4 supporting plate
5 reservoirs
6 memory unit
7 interface
8 transfer unit
9 detection unit
10 analysis unit
11 visualization unit
12 database
13 data processing unit
14 biological specimen
17 insertion of the kit and the biological specimen
18 start of the analysis by the operator
19 transfer of the test records and software to the data processing unit
20 transfer of the respective test records and, if provided, the software to the units 8 to 12
21 taking the markers and process aids from the kit
22 transfer of the markers and process aids to the detection unit
24 analysis of the specimen
25 transfer of the image data
26 analysis of the image data
27 transfer of the analytical data
28 visualization of the analytical data

1. An analyzer for the automated analysis of a biological specimen that comprises
a control system comprising
a data processing unit;
a unit for receiving a kit, wherein the kit comprises a receiving unit with a number of reservoirs for receiving one or more markers required for the analysis of the biological specimen as well as, if needed, the process aids required therefore as well as a memory unit for the electronic storage of one or more electronically stored test records comprising instructions for the performance of the analysis of the biological specimen by means of the markers, and wherein the memory unit is integrated within the receiving unit;
an interface allowing the communication between the data processing unit and the kit;
an interface allowing the communication between the data processing unit and a transfer unit for the transfer of the markers and process aids from the kit to a detection unit;
an interface allowing the communication between the data processing unit and the detection unit for the generation of image data of the biological specimen; and
an interface allowing the communication between the data processing unit and an analysis unit to analyze the image data obtained by means of the detection unit to generate analytical data;
a transfer unit for the transfer of the markers from the kit to a detection unit, wherein the transfer unit communicates via the related interface of the control system therewith;
a detection unit to generate image data of the biological specimen, wherein the detection unit communicates via the related interface of the control system therewith; and
an analysis unit to analyze the image data obtained by means of the detection unit to generate analytical data, wherein the analysis unit communicates via the related interface of the control system therewith.

2. The analyzer according to claim 1,
wherein
further comprising at least one of the following units:
a data base to store image data and analytical data, wherein the data base communicates via the related interface of the control system therewith; and
a visualization unit to visualize image data and analytical data, wherein the visualization unit communicates via the related interface of the control system therewith.

3. The analyzer according to claim 1,
wherein
in the memory unit of the kit
one or more electronically stored test records comprising instructions for performing the analysis of the biological specimen by means of the markers; and optionally
electronically stored software for the evaluation of measuring results obtained from the biological specimen by means of the markers and using the test records;
are stored.

4. The analyzer according to claim 1, wherein
the memory unit of the kit has an interface allowing the communication of the unit for electronic storage with another electronic unit.

5. The analyzer according to claim 1, wherein
the receiving unit of the kit is a microwell plate and/or the memory unit is a storage medium with a hardware interface.

6. The analyzer according to claim 1, wherein
the transfer unit is realized as a microfluidics system.

7. The analyzer according to claim 1, wherein
the receiving unit of the kit is realized as a microfluidics system.

8. The analyzer according to claim 1, wherein
the control system further comprises one or more of the following interfaces:
an interface allowing the communication between the data processing unit and a database for the storage of image data and analytical data; and
an interface allowing the communication between the data processing unit and a visualization unit for the visualization of image data and analytical data.

9. A method for the automated analysis of a biological specimen by means of an analyzer that comprises the steps of:
(a) providing a kit comprising markers, process aids, test records, and optionally software that are required for the analysis of a given biological specimen;
(b) introducing the kit into a control system and transferring test records stored there into a data processing unit of the control system;
(c) determining instructions from test records required for controlling a transfer unit by means of the data processing unit and transferring said instructions via a related interface of the control system to the transfer unit;
(d) determining the instructions from the test records required for controlling a detection unit by means of the data processing unit and transferring said instructions via a related interface of the control system to the detection unit; and
(e) transferring markers and process aids from the kit to the detection unit by means of the transfer unit on the basis of the instructions transferred in step (c); and
(f) generating image data of the biological specimen by means of the detection unit on the basis of the instructions transferred in step (d).

10. The method according to claim 9,
further comprising the following steps:
(g) determining the instructions from the test records required for controlling the analysis unit by means of the data processing unit and transferring said instructions via the related interface of the control system to the analysis unit;
(h) transferring the image data obtained in step (f) to the analysis unit and analyzing the image data by means of the analysis unit using the instructions transferred in step (e) to obtain analytical data.

11. The method according to claim 10,
wherein
in step (g) there is further transferred software that has been transferred from the kit into the data processing unit from the data processing unit to the analysis unit and that in step (h) in addition to the instructions the software for the analysis of the image data is used.

12. The method according to claim 9,
wherein
the image data and analytical data are transferred to the data processing unit and there are stored in the database.

13. The method according to claim 9,
wherein
the image data and analytical data are transferred from the data processing unit via its related interface to a visualization unit.
